(19) United States
(12) Patent Application Publication
(10) Pub. No.: US 2009/0214625 A1
(43) Pub. Date: Aug. 27, 2009

(54) DRUG DELIVERY PATCH

(76) Inventors: Mizuo Nakayama, Tokyo (JP); Akihiko Matsumura, Tokyo (JP); Takehiko Matsumura, Tokyo (JP); Hidero Akiyama, Tokyo (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 11/995,796

(22) PCT Filed: Jul. 18, 2006
(86) PCT No.: PCT/JP2006/314183

§ 371 Date: Mar. 2, 2009

(30) Foreign Application Priority Data
Jul. 15, 2005 (JP) 2005-207675

(51) Int. Cl. A61K 9/70; A61P 43/00

(52) U.S. Cl. 424/449

(57) ABSTRACT

The site to which a patch was stuck may be identified even after the patch is removed.

A patch for drug delivery includes a drug holding portion and a backing layer. The patch further includes a marker capable of indicating a site to which the patch was stuck even after the patch is removed therefrom.

### BACKGROUND

1. Technical Field

The present description relates to a patch for introducing a drug into an organism through a biological interface while stuck to the biological interface.

2. Description of the Related Art

The transdermal introduction of a specific substance, such as a drug, into an organism is well known (see, e.g., FIG. 9). In recent years, transdermally introducing a drug into a body has been stepping into the limelight, along with oral dosage and dosage by injection. This is in part because transdermal delivery does not suffer from certain problems associated with oral dosage and dosage by injection. One problem with oral dosage is that, over the path by which an administered drug reaches a target site (which is often via blood), the drug is decomposed in a digestive organ. As a result, the efficiency of administration is poor, and a large amount of the drug must be administered to exert a sufficient therapeutic effect. One problem with dosage by injection is that it may be painful or distressing for the patient, although the efficiency of administration is high.

The term “patch” may refer to a cloth-like section having adhesive on one surface and including a substance such as a drug or an antigen. In different embodiments, the patch may have a variety of thicknesses and may or may not have an adhesive surface.

As described in, for example, JP 2002-532540 A, a patch containing nicotine as an anti-smoking auxiliary agent may maintain nearly constant drug concentration over a long time period. Such nicotine patches are desirably stuck to a different position each time, to prevent repeated stimulus of a single skin site.

As shown in JP 2005-510488 A, a patch for transdermal delivery may also be used to deliver a local anesthetic, such as morphine hydrochloride. In such cases, the site to which the patch is stuck will also be used for a subsequent treatment.

Thus, in many applications, it may be important to identify the site to which a patch has been stuck even after the patch is removed.

However, once a patch is removed, it is often impossible to accurately identify the site to which the patch was stuck. For example, in the case where a patch is stuck to a site that cannot be observed by a patient, it may be difficult for the patient to later identify the site.

In some cases, the difficulty in identifying the site may be obviated by performing a treatment (such as an injection) immediately after removal of the patch, on the condition that the patient is ready for the treatment immediately after removal. However, the difficulty cannot be completely eliminated, and the person performing the treatment must remember to perform the treatment immediately after removal.

In other situations, when a certain time interval must elapse between the removal of the patch and the next treatment (for example, when a drug is administered at certain time intervals), it can be difficult to positively identify the site to which the patch was stuck.

### BRIEF SUMMARY

The presently disclosed embodiments may allow a doctor or other provider to perform a subsequent treatment at an effective site, and to identify an original site to which a patch for drug delivery was stuck, even after the patch is removed. The patch for drug delivery may include a drug holding portion and a backing layer for introducing a drug into the body of an organism by transdermal delivery when stuck to a biological interface (e.g., skin or mucosa).

According to one embodiment, a patch for introducing a drug into the body of an organism through a biological interface, such as skin or mucosa, may be provided, including a drug holding portion and a backing layer. The patch may introduce the drug by means of transdermal absorption via a surface of the drug holding portion or a membrane in contact with the drug holding portion when the patch is stuck to the organism. The patch may further include a marker configured to indicate a site of the biological interface to which the patch was stuck after the patch is removed from the site.

In one embodiment, the site to which the patch was stuck may thus be positively identified even after the patch is removed.

In one embodiment, the marker may comprise a separable portion of the backing layer or the drug holding portion and may be configured to remain on the biological interface after the patch is removed from the site.

In one embodiment, the site to which the patch was stuck may be identified without introducing a substance separately serving as a marker.

In another embodiment, the marker may comprise a dye or an ink (e.g., a food dye, or ink for printing on a tablet).

In yet another embodiment, the patch may further include at least one electrode connected to an electric power source for actively introducing the drug by iontophoresis. The drug may thus be introduced more quickly.

The term “marker,” as used herein, refers to a substance or component capable of indicating the site to which the patch was stuck, even after the patch is removed, by moving from the side of the patch to the organism when the patch is stuck to the organism. The term “front surface,” as used in the specification including the foregoing description, refers to the surface that is closer to a biological interface during use (e.g., mounting) of a device.

### BRIEF DESCRIPTION OF THE SEVERAL VIEWS OF THE DRAWINGS

In the drawings, identical reference numbers identify similar elements. The sizes and relative positions of elements in the drawings are not necessarily drawn to scale. For example, the shapes of various elements and angles are not drawn to scale, and some of these elements have been arbitrarily enlarged and positioned to improve drawing legibility. Further, the particular shapes of the elements as drawn are not intended to convey any information regarding the actual shape of the particular elements and have been solely selected for ease of recognition in the drawings.

FIG. 1 shows a patch for drug delivery, according to one illustrated embodiment.

FIG. 2 is a cross-sectional view of the patch of FIG. 1, taken along the line II-II.

FIG. 3 is a cross-sectional view of a patch for drug delivery, according to another illustrated embodiment.

FIG. 4A shows a patch for drug delivery, before the patch is stuck, according to one illustrated embodiment.

FIG. 4B shows the patch of FIG. 4A stuck to a site on an organism.

FIG. 4C shows the patch of FIG. 4A being removed.

FIG. 4D shows the site, after the patch of FIG. 4A has been removed.

FIG. 5 shows an example site for the patch of FIG. 4A as the patch is being removed.

FIG. 6 is a schematic block diagram showing an iontophoresis device, according to one illustrated embodiment.

FIG. 7 is an enlarged view of a portion V of the iontophoresis device of FIG. 6.

FIG. 8 is a schematic block diagram of an iontophoresis device, according to another illustrated embodiment.

FIG. 9 shows a conventional patch for drug delivery.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures, methods and compositions associated with patches, drug delivery and iontophoresis have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described may be included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the disclosure clearly dictates otherwise.

The headings and Abstract of the Disclosure provided herein are for convenience only and do not interpret the scope or meaning of the embodiments.

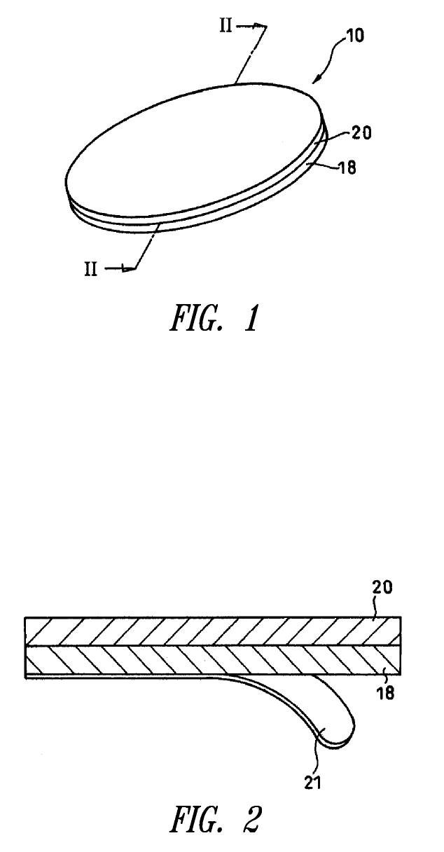

FIGS. 1 and 2 show a patch 10 for drug delivery, according to one embodiment. FIG. 1 is a perspective view of the patch 10, and FIG. 2 is a cross-sectional view taken along the line II-II of FIG. 1.

The patch 10 may comprise a drug holding portion (or layer) 18 holding a substance (e.g., a drug) to be introduced through a biological interface, such as an anesthetic, and a backing layer 20 adjacent the drug holding portion 18. A front side of the drug holding portion 18 (not visible in FIG. 1) may be configured to be stuck to a biological interface of an organism.

The front surface of the drug holding portion 18 may be provided with a film-like removable layer 21 to be removed before sticking the patch 10 to a biological interface.

The backing layer 20 may function as a primary constituent of the patch 10 and may impart flexibility and coverage to the patch 10. The material used for the backing layer 20 may, in one embodiment, be therapeutically inactive and may be configured so as not to absorb the drug of a drug composition in the patch 10 or any other component, such as a stabilizer. The backing layer 20, serving as a protective cover, may be made from one or more soft sheets or films, thereby allowing the patch 10 to follow the outline of a biological interface, such as skin. The backing layer 20 may also be permeable to air, vapor, etc. A backing layer 20 having such permeability may enable respiration (i.e., an exchange of oxygen and carbon dioxide) at the site of a biological interface where the patch is stuck, and may also enable the exchange of water vapor from the surface of the biological interface.

In one embodiment, the backing layer 20 may comprise a copolyester, a polyether/polyamide copolymer, polyurethane, or a polyethylene derivative. Of course, the material for the backing layer 20 is not limited to the above. A suitable example of the polyether/polyamide copolymer is PEBAX®. A suitable example of polyurethane is ESTANE. A suitable example of a polyethylene derivative is a SKYCARE AND SCYAIR film.

The drug holding portion 18 may, in one embodiment, comprise a hydrogel. Any hydrogel may be used for the patch 10, including those that can be subjected to γ-ray sterilization and that can be impregnated with local anesthetics. The hydrogel may, in one embodiment, have sufficient adhesiveness to allow the patch 10 to adhere to an application site and be removed with little if any discomfort or damage to a wound.

The hydrogel may comprise polyvinyl pyrrolidone (PVP) crosslinked by means of an electron beam and having an average molecular weight of about 500,000 daltons to about 2,000,000 daltons (more preferably about 900,000 daltons to about 1,500,000 daltons). In one embodiment, the hydrogel may contain crosslinked polyvinyl pyrrolidone, a local anesthetic, and water. Of course, a stabilizer or other components may also be added to the hydrogel.

Many suitable hydrogels are commercially available. For example, a suitable hydrogel can be purchased from Hydrogel Design Systems, or Tyco, Inc.

Any drug that may be introduced through a biological interface to exert a therapeutic or medical effect may be applied using the patch 10. Specific examples of such drugs are listed below. Of course, potential drugs are not limited to any of the following.

(A) A coronary vasodilator, such as nitroglycerin or isosorbide nitrate;

(B) A hypotensive agent, such as clonidine hydrochloride or nifedipine;

(C) An anesthetic painkiller, such as morphine hydrochloride, lidocaine hydrochloride, or fentanyl citrate;

(D) An anti-inflammatory painkiller, such as ketoprofen, indomethacin, ketorolac, loxoprofen, tenidap, or buprenorfen hydrochloride;

(E) A bronchodilator, such as isoproterenol sulfate, salibutamol sulfate, or tulobuterol hydrochloride;

(F) An androgen, such as testosterone propionate or fluoxymesterone;

(G) An estrogen, such as estradiol benzoate, ethinylestradiol, or estriol;

(H) A progesterone, such as progesterone, norethisterone, or levonorgestrel;

(I) An antiallergic drug, such as sodium cromoglycate, azelastine, or ketotifen fumarate;

(J) A muscle relaxant, such as eberison hydrochloride or afloqualone;

(K) An antihistamine agent, such as diphenhydramine or d1-chlorpheniramine maleate;

(L) A general anesthetic, such as thiopental sodium or ketamine hydrochloride;

(M) An antitussive or expectorant agent, such as codeine phosphate or ephedrine hydrochloride;

(N) An antismoking auxiliary agent, such as nicotine; or

(O) A vitamin agent, such as ascorbic acid.

Two or more kinds of the above drugs may also be used in combination, as desired. In addition, any one of the drugs may be sealed in the drug holding portion 18 in the form of a compound derived to an ester body, a compound derived to an amino body, a compound derived to an acetal body, or a medically acceptable inorganic or organic salt.

In one embodiment, the marker used in the embodiment of FIG. 1 may be configured so as not to chemically react with a drug administered to a patient or to harm the patient. The marker may be one that can be visually observed under normal lighting conditions, such as a fat dye, an aqueous dye, or an aqueous pigment. In another embodiment, the marker may comprise fluorescent dyestuff that may be visualized under ultraviolet light, or may be a substance, such as luciferin, that is bound to oxygen owing to the action of a specific enzyme, such as luciferase, to thereby fluoresce. In yet another embodiment, the marker may be identified by an odor or flavor, in addition to, or instead of, visual cues.

In one embodiment, a food dye may be employed as a marker. For example, a synthetic dyestuff, such as amaranth (Food Red No. 2), erythrosine (Food Red No. 3), Allura Red AC (Food Red No. 40), new coccin (Food Red No. 102), rose bengal (Food Red No. 105), acid red (Food Red No. 106), Tartrazine (Food Yellow No. 4), sunset yellow FCF (Food Yellow No. 5), brilliant blue FCF (Food Blue No. 1), or indigocarmine (Food Blue No. 2), or a natural dyestuff, such as a cochineal dyestuff or an annatto dyestuff, may be used.

In another embodiment, inks such as Opacode®, Opacode WB®, or NT23BR available from Colorcon may be used as a marker. Such inks are already used for printing on, for example, oral medicine or oral medicine capsules.

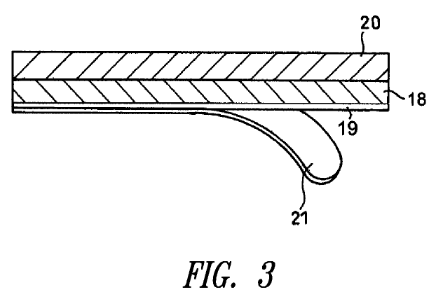

In one embodiment, the marker may be sealed in the drug holding portion 18, mixed with the drug. Alternatively, as shown in FIG. 3, the marker may be introduced into a layer, such as marker layer 19, formed separately from the drug holding portion 18.

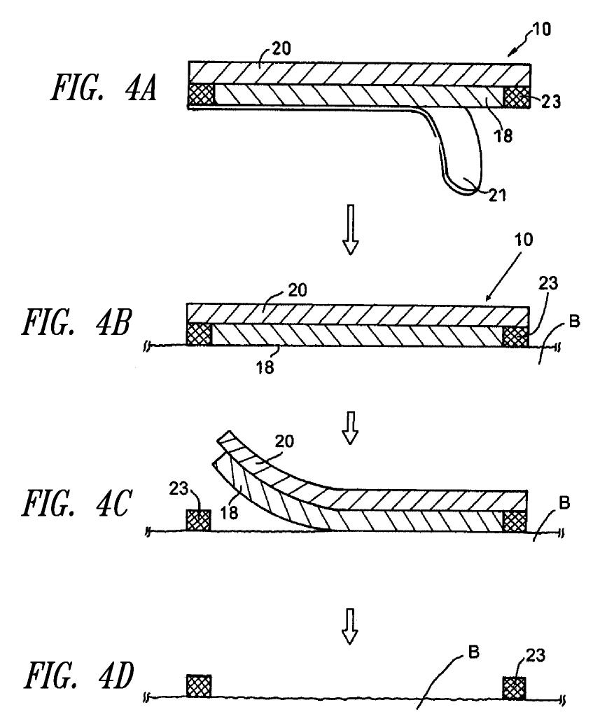

In another embodiment, as shown in FIG. 4A, one portion 23 of the backing layer 20 may be separable, such that it remains on the biological interface when the patch 10 is removed. Thus, the remaining portion 23 may function as a marker. In such an embodiment, the site to which the patch 10 has been stuck may be identified without introduction of a separate substance to serve as a marker.

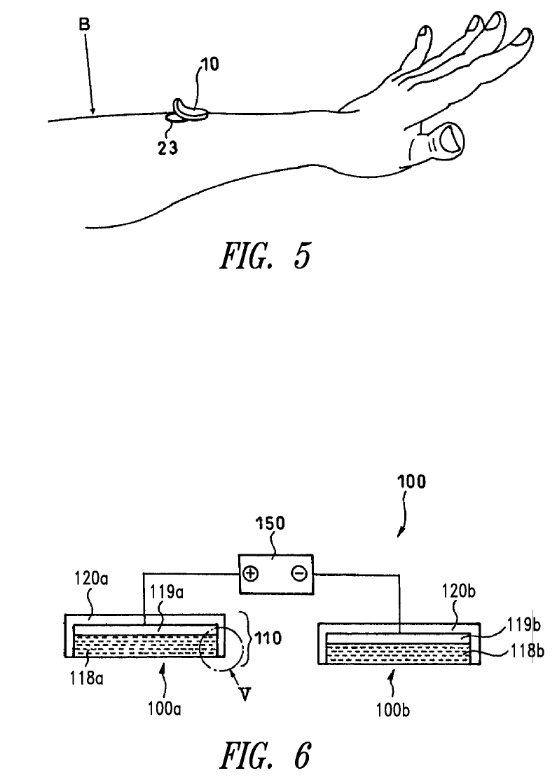

As shown in FIGS. 4B, 4C and 4D, after the patch 10 has been stuck to the biological interface and removed, only the remaining portion 23 may remain on the side of the organism. The site to which the patch 10 was stuck may then later be identified, as illustrated in FIG. 5. That is, the patch 10 may be configured such that an adhesive strength between the remaining portion 23 and a side of the organism is greater than that between the remaining portion 23 and the backing layer 20 and that between the remaining portion 23 and the drug holding portion 18.

When the patch 10 for drug delivery is stuck to a biological interface (such as a skin or mucosa), the marker itself may also contact and move to the side of the organism. Thus, the site to which the patch 10 was stuck may be accurately identified even after the patch 10 is removed.

In one embodiment, the marker (whether a dye or a separable component of the backing layer 20) may include a substance that changes its color (e.g., becoming colorless) after a certain time period. In addition, a substance may be used that changes its color when the concentration of an administered drug is equal to or lower than a certain concentration, enabling the place to which the patch was stuck to be accurately identified and an accurate timing at which the patch should be stuck to be identified.

From a physiological viewpoint, use of such a marker may be particularly effective when a certain time interval (or longer) should elapse between drug administrations, or when a subsequent drug administration must be performed within a certain time period in order to continuously administer a drug.

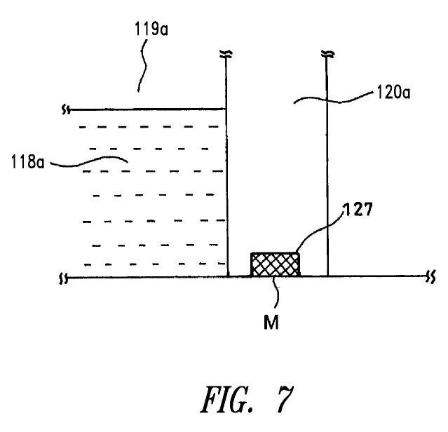

FIGS. 6-8 show the application of patches that may be used as electrodes for iontophoresis devices, according to different embodiments.

An iontophoresis device may include a working patch having a drug holding portion holding a drug (e.g., an ionic drug) and a non-working patch used as a counter electrode of the working patch. The iontophoresis device may electrically drive the drug into an organism by applying a voltage having the same polarity as that of a drug ion in a drug holding portion to the working patch when both patches are brought into contact with a biological interface, thereby actively transferring the drug to the organism. Such iontophoresis devices may reduce the pain of drug administration, may allow drug administration without an initial passage effect, and may enable electrical control of the amount of drug administered.

FIG. 6 is a block diagram schematically showing an iontophoresis device 100. FIG. 7 is an enlarged view of a portion V of the iontophoresis device 100 of FIG. 6.

The iontophoresis device 100 may include a working patch 100a and a ground patch (e.g., a non-working patch) 100b connected to an electric power source 150. In the illustrated example, the working patch 100a is connected to a positive terminal (anode), and the ground patch 100b is connected to a negative terminal (cathode). For convenience of description, an iontophoresis device for administering a drug whose drug component dissociates to positive drug ions (for example, lidocaine hydrochloride or morphine hydrochloride, both as anesthetics) is described in detail herein. However, in other embodiments, iontophoresis devices for administering a drug whose drug component dissociates to negative drug ions (for example, ascorbic acid as a vitamin agent) may also be used, for example, by reversing the polarity of a voltage applied to the electrodes and the polarity of an exchange group introduced into an ion exchange membrane or an ion exchange resin.

As shown in FIG. 6, the working patch 100a may comprise: an electrode 119a connected to the electric power source 150; a drug holding portion 118a holding a drug, the drug holding portion 118a in contact with the electrode 119a and energized via the electrode 119a; and a container 120a housing them. The ground patch 100b may comprise: an electrode 119b; an electrolyte solution holding portion 118b holding an electrolyte solution, the electrolyte solution holding portion 118b in contact with the electrode 119b and energized via the electrode 119b; and a container 120b housing them. In one embodiment, the container 120a may serve as a backing layer.

The electrodes 119a and 119b may comprise any conductive material. An active electrode, such as a silver/silver chloride couple electrode, capable of suppressing the generation of H⁺ or OH⁻ ions due to the electrolysis of water, may also be used.

The drug holding portion 118a may hold a solution (i.e., a drug solution) whose drug component dissociates to positive drug ions as a result of dissolution.

A marker may, in one embodiment, be sealed in the drug holding portion 118a together with the drug. Of course, a divided marker holding portion (not shown) may also be arranged in the drug holding portion 118a.

As shown in FIG. 7, the marker may also be arranged in, for example, a recess 127 at a surface of the container 120a brought into contact with the biological interface. Such an arrangement may also be used for an iontophoresis device 200, described in greater detail below.

The electrolyte solution holding portion 118b may hold an electrolyte solution for maintaining energization. Phosphate buffered saline or physiological saline may be used as the electrolyte solution. In another embodiment, the generation of gas due to the electrolysis of water and an accompanying increase in conductive resistance or a fluctuation in pH value may be mitigated or prevented by using an electrolyte that is oxidized or reduced more easily than water. For example, the electrolyte solution may comprise: inorganic compounds (e.g., ferrous phosphate and ferric phosphate); medical agents (e.g., ascorbic acid (vitamin C) and sodium ascorbate); organic acids (e.g., hydrochloric acid, oxalic acid, malic acid, succinic acid, and fumaric acid and/or salts thereof), or a mixture of the above.

Each of the drug holding portion 118a and the electrolyte solution holding portion 118b may hold a corresponding solution in a liquid state. Alternatively, each of the drug holding portion 118a and the electrolyte solution holding portion 118b may hold a corresponding solution in an impregnated fibrous sheet (for example, gauze or filter paper), or in another material having the ability to retain water (for example, a polymer gel sheet, such as the above-described hydrogel), so that the solution is more easily handled.

The iontophoresis device 100 may be used with the working patch 100a and the ground patch 100b each stuck to a biological interface of an organism. The ground patch 100b may, in one embodiment, be placed at a site on the biological interface (e.g., skin, mucosa, etc.) within a certain distance from the working patch 100a.

When the electric power source 150 is turned on, a positive voltage and a negative voltage may be applied to the electrodes 119a and 119b, respectively. Drug ions in the drug holding portion 118a may then be driven by the voltage toward the organism. A drug may thus be actively introduced into the organism by means of the iontophoresis device 100, and the drug may be more quickly absorbed and allowed to permeate into the organism.

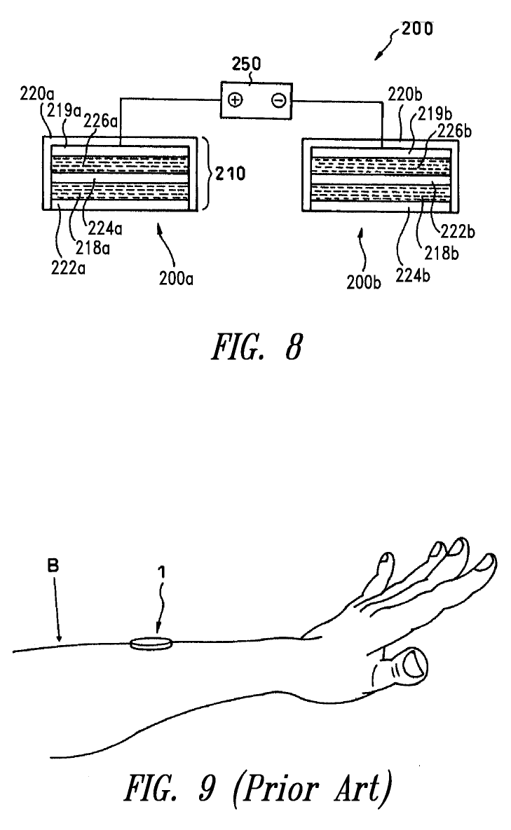

Another iontophoresis device 200 is illustrated in FIG. 8. The iontophoresis device 200 may be formed by adding components to the iontophoresis device 100. Reference numerals having the same lower two digits as those of the reference numerals of the iontophoresis device 100 are given to components of the iontophoresis device 200 that are similar or identical to those of the iontophoresis device 100, and duplicate description is omitted.

Each of a working patch 200a and a ground patch 200b of the iontophoresis device 200 may have multiple ion exchange membranes. The working patch 200a may comprise an electrode 219a connected to an electric power source 250, a buffer solution holding portion 226a, an anion exchange membrane 224a, a drug holding portion 218a, and a cation exchange membrane 222a. These components may be housed in a container 220a (which may serve as a backing layer) and may approach the surface of the organism in the above order. The ground patch 200b may comprise an electrode 219b, a buffer solution holding portion 226b, a cation exchange membrane 222b, an electrolyte solution holding portion 218b, and an anion exchange membrane 224b. These components may be housed in a container 220b and may approach the surface of the organism in the above order.

Each of the anion exchange membranes 224a, 224b may include an ion exchange membrane for permitting passage of negative ions. For example, an anion exchange membrane such as a NEOSEPTA (AM-1, AM-3, AMX, AHA, ACH, or ACS) manufactured by Tokuyama Co., Ltd may be used for each of the anion exchange membranes 224a, 224b.

In one embodiment, the anion exchange membranes 224a, 224b may comprise a semi-permeable film including a polyolefin resin, a vinyl chloride-based resin, a fluorine-based resin, a polyamide resin, or a polyimide resin, having cavities of which a whole or a part are filled with an anion exchange resin. In such an embodiment, the cavities of the film may be filled with the anion exchange resin by: impregnating the cavities of the porous film with a solution prepared by blending a crosslinkable monomer such as styrene-divinylbenzene or chloromethylstyrene-divinylbenzene with a polymerization initiator; polymerizing the resultant; and introducing into the polymer an anion exchange group, such as a primary amino group, a secondary amino group, a tertiary amino group, a quaternary ammonium group, a pyridyl group, an imidazole group, a quaternary pyridinium group, or a quaternary imidazolium group. Other anion exchange membranes may also be used in other embodiments.

Each of the cation exchange membranes 222a, 222b may include an ion exchange membrane for permitting passage of positive ions. For example, a cation exchange membrane such as a NEOSEPTA (CM-1, CM-2, CMX, CMS, or CMB) manufactured by Tokuyama Co., Ltd may be used for each of the cation exchange membranes 222a, 222b.

In one embodiment, the cation exchange membranes 222a, 222b may comprise a semi-permeable film including a polyolefin resin, a vinyl chloride-based resin, a fluorine-based resin, a polyamide resin, or a polyimide resin, having cavities of which a whole or a part are filled with a cation exchange resin. In such an embodiment, the cavities of the film may be filled with the cation exchange resin by: impregnating the cavities of the porous film with a solution prepared by blending a crosslinkable monomer such as styrene-divinylbenzene or chloromethylstyrene-divinylbenzene with a polymerization initiator; polymerizing the resultant; and introducing into the polymer a cation exchange group, such as a sulfonic group, a carboxylic group, or a phosphoric group. Other cation exchange membranes may also be used in other embodiments.

In the iontophoresis device 200, the movement of a positive ion having a small molecular weight from the buffer solution holding portion 226a to the drug holding portion 218a may be inhibited by the anion exchange membrane 224a. In addition, the movement of a negative ion having a small molecular weight from the side of the organism to the drug holding portion 218a may be inhibited by the cation exchange membrane 222a. As a result, decomposition of the drug at the electrode 219a and fluctuations in pH at the biological interface may be suppressed, improving the stability of drug administration.

In one embodiment, as illustrated, the drug holding portion 218a and the buffer solution holding portion 226a may be separated from each other. Thus, the generation of gases and corresponding fluctuations in pH may be mitigated by: blending the electrolyte solution of each of the buffer solution holding portions 226a and 226b with a substance having an oxidation-reduction potential lower than that of water; and causing multiple ion species to be present in the buffer solutions.

In one embodiment, carbon or an inactive metal (e.g., platinum) may be used for each of the electrodes 219a and 219b. In particular, a composite carbon electrode may be used, including: a terminal member prepared by blending a polymer matrix which has high conductivity and flexibility and which does not allow a metal ion to be eluted with a carbon powder; and a conductive sheet composed of carbon fiber or carbon fiber paper, or a conductive sheet impregnated with a polymer elastomer.

In the illustrated embodiment, the marker may be sealed in the drug holding portion 218a. However, the presence of the cation exchange membrane 222a should be taken into consideration. If a dyestuff component of the marker dissociates to cations, the marker may be sealed in the drug holding portion 218a. If not, the marker may be unable to move to the side of the organism when the patch is stuck thereto, and the marker will not be able to provide the function of indicating the place to which the patch was stuck.

In another embodiment, the marker may be applied to and disposed on the front surface (i.e., the surface in contact with the organism) of the cation exchange membrane 222a. Alternatively (though not shown), the marker may be placed in a recess arranged on a part of a surface of the container 220a in contact with the organism instead of being sealed in the drug holding portion 218a.

Using the above arrangements, in each of the iontophoresis devices 100, 200, when a patch is stuck to an organism in order to perform iontophoresis, a marker may move to the side of the organism, whereby a site to which the patch was stuck may be accurately identified even after the patch is removed.

The above-described embodiments may be widely applied to patches for transdermal delivery, including those for use in the medical field.

### DESCRIPTION OF REFERENCE NUMERALS

10 Patch for Drug Delivery
18 Drug Holding Portion (Layer)
20 Backing Layer
21 Removable Layer
23 Remaining Portion

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

The various embodiments described above can be combined to provide further embodiments. From the foregoing it will be appreciated that, although specific embodiments have been described herein for purposes of illustration, various modifications may be made without deviating from the spirit and scope of the teachings. Accordingly, the claims are not limited by the disclosed embodiments.

1. A patch for introducing a drug through a biological interface, comprising:
a drug holding portion configured to hold the drug;
a backing layer adjacent a surface of the drug holding portion; and
a marker configured to indicate a site of the biological interface to which the patch was stuck after the patch is removed from the site.

2. The patch of claim 1, wherein the marker comprises a separable portion of the backing layer or the drug holding portion, and is configured to remain on the biological interface after the patch is removed from the site.

3. The patch of claim 1, wherein the marker comprises a dye or an ink.

4. The patch of claim 1, further comprising:
at least one electrode connected to an electric power source to introduce the drug by iontophoresis.

5. The patch of claim 1, further comprising:
an electric power source; and
at least one electrode electrically coupled to the electric power source to iontophoretically deliver the drug.

6. The patch of claim 1, wherein the drug holding portion is a hydrogel.

7. The patch of claim 1, wherein the marker does not chemically react with the drug.

8. The patch of claim 1, wherein the marker is visually discernable when illuminated by white light.

9. The patch of claim 1, wherein the marker is visually discernable only when illuminated by ultraviolet light.

10. The patch of claim 1, wherein the marker is a food dye.

11. The patch of claim 1, wherein the marker is an ink.

12. The patch of claim 1, wherein the marker is luciferin.

13. The patch of claim 1, wherein the marker is odorous.

14. The patch of claim 1, wherein the marker is flavored.
